(19) United States
(12) Patent Application Publication
(10) Pub. No.: US 2013/0158638 A1
(43) Pub. Date: Jun. 20, 2013

(54) STIMULATION LEAD AND METHOD OF FABRICATION

(71) Applicants: Enri Zhulati, Fort Worth, TX (US); Thomas Wright, Dallas, TX (US)

(72) Inventors: Enri Zhulati, Fort Worth, TX (US); Thomas Wright, Dallas, TX (US)

(73) Assignee: ADVANCED NEUROMODULATION SYSTEMS, INC., Plano, TX (US)

(21) Appl. No.: 13/608,914

(22) Filed: Sep. 10, 2012

Related U.S. Application Data
(60) Provisional application No. 61/558,900, filed on Nov. 11, 2011.
(60) Provisional application No. 61/558,904, filed on Nov. 11, 2011.
(60) Provisional application No. 61/558,909, filed on Nov. 11, 2011.

(51) Int. Cl. A61N 1/05; H01B 13/00

(52) U.S. Cl. CPC A61N 1/0553; H01B 13/00
USPC 607/116; 29/825

(57) ABSTRACT

In one embodiment, an implantable stimulation lead for delivering electrical stimulation to tissue of a patient, the implantable stimulation lead comprises: a lead body comprising a plurality of wires strands that form a solid helical hollow tube that is maintained by mutual contact pressure of neighboring wire strands, wherein the plurality of wire strands comprise an outer coating of non-conductive material to electrically isolate each of the plurality of wire strands from each other; and a plurality of electrodes with each electrode being electrically coupled to one of the plurality of wire strands.

### TECHNICAL FIELD

This application is generally related to implantable stimulation leads for delivering electrical pulses to tissue of a patient and methods of fabrication.

### BACKGROUND

Neurostimulation systems are devices that generate electrical pulses and deliver the pulses to nerve tissue to treat a variety of disorders. Spinal cord stimulation (SCS) is the most common type of neurostimulation within the broader field of neuromodulation. In SCS, electrical pulses are delivered to nerve tissue in the spine typically for the purpose of chronic pain control. While a precise understanding of the interaction between the applied electrical energy and the nervous tissue is not fully appreciated, it is known that application of an electrical field to spinal nervous tissue can effectively mask certain types of pain transmitted from regions of the body associated with the stimulated nerve tissue. Specifically, applying electrical energy to the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain.

SCS systems generally include a pulse generator and one or more leads. A stimulation lead includes a lead body of insulative material that encloses wire conductors. The distal end of the stimulation lead includes multiple electrodes that are electrically coupled to the wire conductors. The proximal end of the lead body includes multiple terminals (also electrically coupled to the wire conductors) that are adapted to receive electrical pulses. The distal end of a respective stimulation lead is implanted within the epidural space to deliver the electrical pulses to the appropriate nerve tissue within the spinal cord that corresponds to the dermatome(s) in which the patient experiences chronic pain. The stimulation leads are then tunneled to another location within the patient's body to be electrically connected with a pulse generator or, alternatively, to an “extension.”

The pulse generator is typically implanted within a subcutaneous pocket created during the implantation procedure. In SCS, the subcutaneous pocket is typically disposed in a lower back region, although subclavicular implantations and lower abdominal implantations are commonly employed for other types of neuromodulation therapies.

The pulse generator is typically implemented using a metallic housing that encloses circuitry for generating the electrical pulses, control circuitry, communication circuitry, a rechargeable battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on a stimulation lead.

### SUMMARY

In one embodiment, an implantable stimulation lead for delivering electrical stimulation to tissue of a patient, the implantable stimulation lead comprises: a lead body comprising a plurality of wires strands that form a solid helical hollow tube that is maintained by mutual contact pressure of neighboring wire strands, wherein the plurality of wire strands comprise an outer coating of non-conductive material to electrically isolate each of the plurality of wire strands from each other; and a plurality of electrodes with each electrode being electrically coupled to one of the plurality of wire strands.

The foregoing has outlined rather broadly certain features and/or technical advantages in order that the detailed description that follows may be better understood. Additional features and/or advantages will be described hereinafter which form the subject of the claims. It should be appreciated by those skilled in the art that the conception and specific embodiments disclosed may be readily utilized as a basis for modifying or designing other structures for carrying out the same purposes. It should also be realized by those skilled in the art that such equivalent constructions do not depart from the spirit and scope of the appended claims. The novel features, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the appended claims.

### BRIEF DESCRIPTION OF THE DRAWINGS

FIG. 1 depicts a stimulation system according to one representative embodiment.

FIGS. 2A-2C respectively depict stimulation portions for inclusion at the distal end of a lead according to some representative embodiments.

FIG. 3 depicts a lead body with a hollow helical tube of multiple wire strands according to one representative embodiment.

FIG. 4 depicts the lead body of FIG. 3 in a cross-sectional view.

FIG. 5 depicts a wire strand with an outer insulator and highly conductive core according to one representative embodiment.

FIG. 6 depicts a lead body with electrodes fabricated thereon according to one representative embodiment.

FIG. 7 depicts a system for fabricating a helical hollow tube from wire strands according to one representative embodiment.

FIG. 8 depicts another lead body for use in implantable stimulation leads according to one representative embodiment.

### DETAILED DESCRIPTION

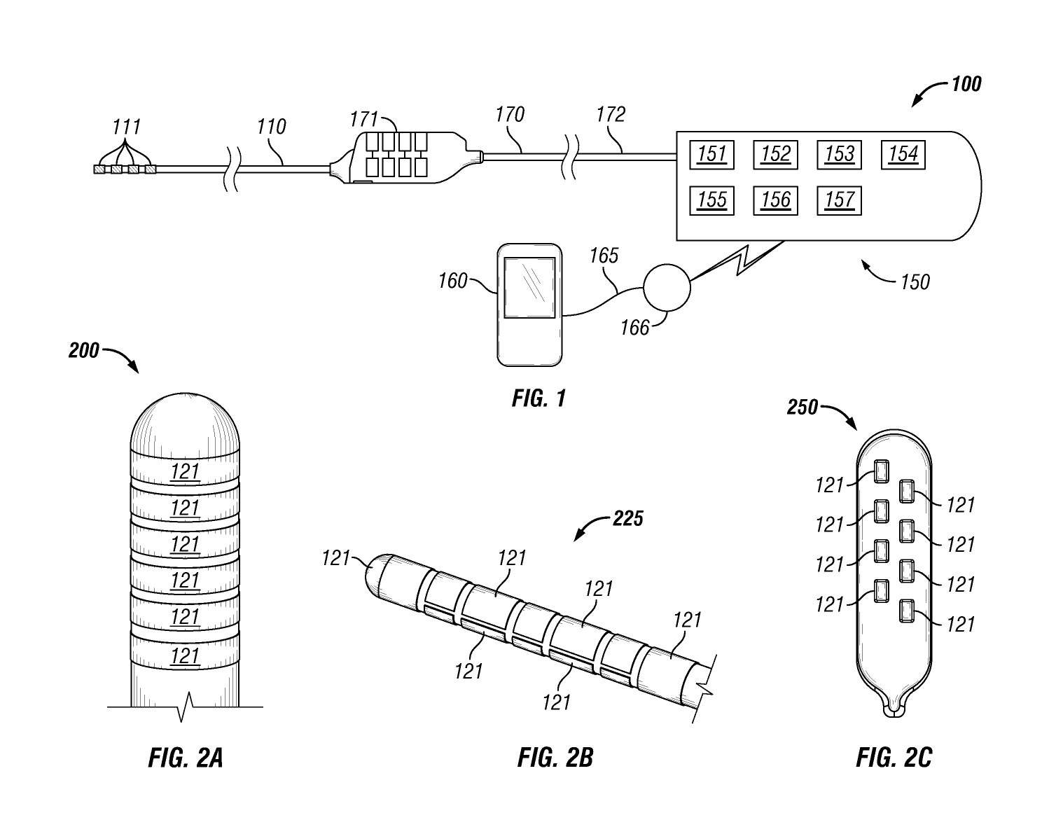

FIG. 1 depicts stimulation system 100 that generates electrical pulses for application to tissue of a patient according to one embodiment. System 100 includes implantable pulse generator 150 that is adapted to generate electrical pulses for application to tissue of a patient. For example, system 100 may be adapted to stimulate spinal cord tissue, peripheral nerve tissue, deep brain tissue, cortical tissue, cardiac tissue, digestive tissue, pelvic floor tissue, or any other suitable tissue within a patient's body.

Implantable pulse generator 150 may comprise a metallic housing that encloses controller 151, pulse generating circuitry 152, charging coil 153, battery 154, far-field and/or near field communication circuitry 155, battery charging circuitry 156, switching circuitry 157, etc. of the device. Controller 151 typically includes a microcontroller or other suitable processor for controlling the various other components of the device. Software code is typically stored in memory of the pulse generator 150 for execution by the microcontroller or processor to control the various components of the device.

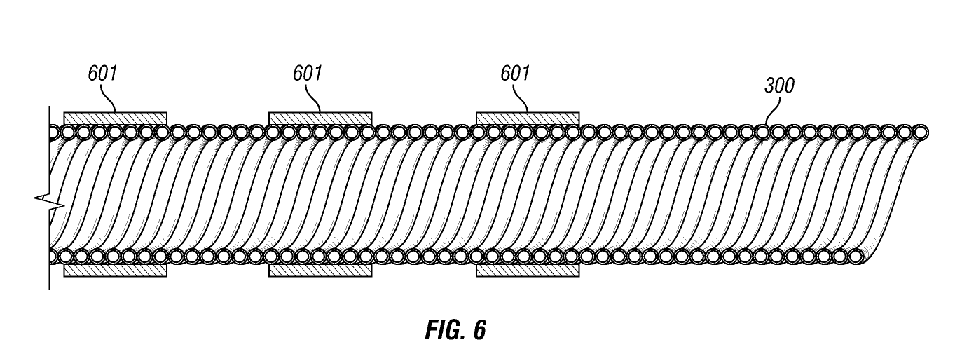

Pulse generator 150 may comprise one or more attached extension components 170 or be connected to one or more separate extension components 170. Alternatively, one or more stimulation leads 110 may be connected directly to pulse generator 150. Within pulse generator 150, electrical pulses are generated by pulse generating circuitry 152 and are provided to switching circuitry 157. The switching circuit connects to output wires, traces, lines, or the like (not shown in FIG. 3) which are, in turn, electrically coupled to internal conductive wires (not shown in FIG. 3) of lead body 172 of extension component 170. The conductive wires, in turn, are electrically coupled to electrical connectors (e.g., “Bal-Seal” connectors) within connector portion 171 of extension component 170. The terminals of one or more stimulation leads 110 are inserted within connector portion 171 for electrical connection with respective connectors. Thereby, the pulses originating from pulse generator 150 and conducted through the conductors of lead body 172 are provided to stimulation lead 110. The pulses are then conducted through the conductors of lead 110 and applied to tissue of a patient via electrodes 111. Any suitable known or later developed design may be employed for connector portion 171.

For implementation of the components within pulse generator 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

An example and discussion of “constant current” pulse generating circuitry is provided in U.S. Patent Publication No. 20060170486 entitled “PULSE GENERATOR HAVING AN EFFICIENT FRACTIONAL VOLTAGE CONVERTER AND METHOD OF USE,” which is incorporated herein by reference. One or multiple sets of such circuitry may be provided within pulse generator 150. Different pulses on different electrodes may be generated using a single set of pulse generating circuitry using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 may comprise a lead body comprising a plurality of conductors that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number of electrodes 111, terminals, and internal conductors.

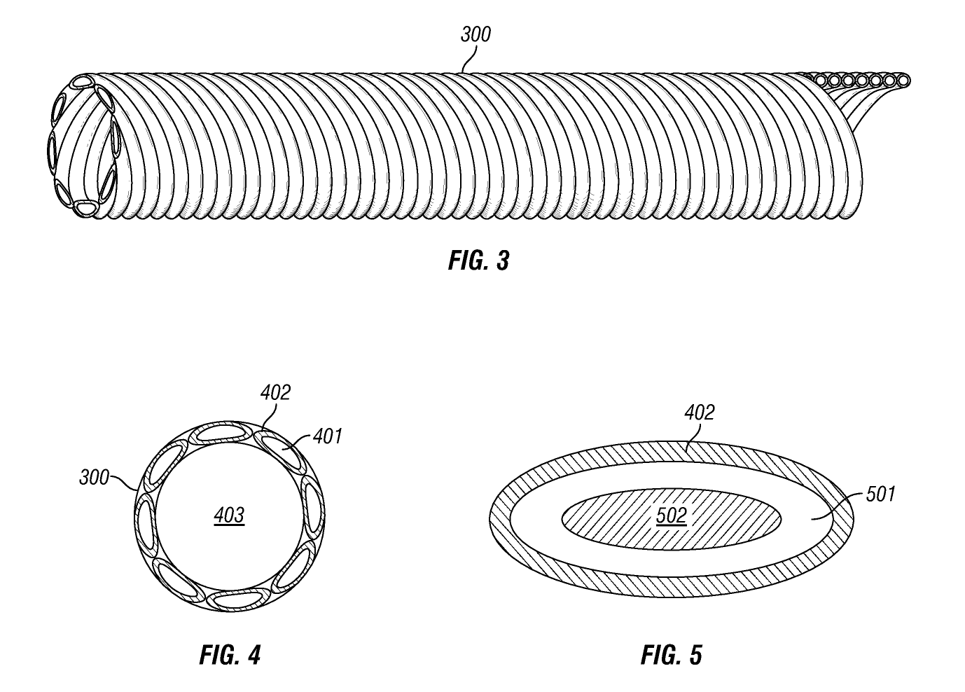

FIGS. 2A-2C respectively depict stimulation portions 200, 225, and 250 for inclusion at the distal end of lead 110. Stimulation portion 200 depicts a conventional stimulation portion of a “percutaneous” lead with multiple ring electrodes. Stimulation portion 225 depicts a stimulation portion including several “segmented electrodes.” The term “segmented electrode” is distinguishable from the term “ring electrode.” As used herein, the term “segmented electrode” refers to an electrode of a group of electrodes that are positioned at the same longitudinal location along the longitudinal axis of a lead and that are angularly positioned about the longitudinal axis so they do not overlap and are electrically isolated from one another. Example fabrication processes are disclosed in U.S. Patent Publication No. 2010072657, entitled, “METHOD OF FABRICATING STIMULATION LEAD FOR APPLYING ELECTRICAL STIMULATION TO TISSUE OF A PATIENT,” which is incorporated herein by reference. Stimulation portion 250 includes multiple planar electrodes on a paddle structure.

The lead bodies of lead(s) 110 and extension component 170 may be fabricated with interior wire strands wound together to form a hollow helical tube as will be further discussed below. The hollow helical tube may permit leads 110 and extension component 170 to be relatively mechanically robust against repetitive bending and elongation forces. For example, the helical hollow tube may provided greater tensile strength and cause the lead to be less susceptible to conductor factures than some known stimulation leads. Also, the hollow helical tube may permit the outer diameter of the lead bodies of lead 110 and extension component 170 to be relatively small. Although these characteristics are possible using helical hollow tubes, these characteristics are not deemed essential or critical for leads 110 and component 170.

Controller device 160 may be implemented to recharge battery 154 of pulse generator 150 (although a separate recharging device could alternatively be employed). A “wand” 165 may be electrically connected to controller device through suitable electrical connectors (not shown). The electrical connectors are electrically connected to coil 166 (the “primary” coil) at the distal end of wand 165 through respective wires (not shown). Typically, coil 166 is connected to the wires through capacitors (not shown). Also, in some embodiments, wand 165 may comprise one or more temperature sensors for use during charging operations.

The patient then places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. Controller 160 generates an AC-signal to drive current through coil 166 of wand 165. Assuming that primary coil 166 and secondary coil are suitably positioned relative to each other, the secondary coil is disposed within the field generated by the current driven through primary coil 166. Current is then induced in secondary coil. The current induced in the coil of the implantable pulse generator is rectified and regulated to recharge battery 154 by charging circuitry 156. Charging circuitry 156 may also communicate status messages to controller 160 during charging operations using pulse-loading or any other suitable technique. For example, controller 160 may communicate the coupling status, charging status, charge completion status, etc.

External controller device 160 is also a device that permits the operations of pulse generator 150 to be controlled by user after pulse generator 150 is implanted within a patient, although in alternative embodiments separate devices are employed for charging and programming. Also, multiple controller devices may be provided for different types of users (e.g., the patient or a clinician). Controller device 160 can be implemented by utilizing a suitable handheld processor-based system that possesses wireless communication capabilities. Software is typically stored in memory of controller device 160 to control the various operations of controller device 160. Also, the wireless communication functionality of controller device 160 can be integrated within the handheld device package or provided as a separate attachable device. The interface functionality of controller device 160 is implemented using suitable software code for interacting with the user and using the wireless communication capabilities to conduct communications with IPG 150.

Controller device 160 preferably provides one or more user interfaces to allow the user to operate pulse generator 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc. IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 01/93953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference.

Example commercially available neurostimulation systems include the EON MINI™ pulse generator and RAPID PROGRAMMER™ device from St. Jude Medical Neuromodulation Division (Plano, Tex.). Other commercially available systems include the PRIMEADVANCED™ and RESTORE™ available from Medtronic, Inc. (Minneapolis, Minn.) and the PRECISION™ neurostimulation system available from Boston Scientific Neuromodulation Corp. (Valencia, Calif.).

FIG. 3 depicts lead body 300 according to one representative embodiment. Lead body 300 is formed in a manner that generates a wire-stranded hollow tube. A plurality of metallic wires are preformed with a predetermined forming rate and twisted along a circular line into a hollow configuration. By forming and twisting of the metallic wires, neighboring metallic elements are subjected to compression and are disposed in a configuration without gaps between the neighboring metallic wires. Further, the contact pressure between the neighboring metallic wires attains sufficient tightness, circularity, diametrical uniformity, and mechanical integrity to prevent the wire-stranded tube formed by the wires from inadvertently collapsing. The contact pressure maintains the helical hollow tube configuration without requiring welding, adhesives, or other supplemental securing means between the various wire strands. Details regarding formation of helical hollow tubes may be found in U.S. Pat. No. 6,881,194 and laid-open Japanese Patent Application No. 11-25758 which are incorporated herein by reference.

FIG. 4 depicts a cross-sectional view of lead body 300 according to one representative embodiment. The multiple wires of lead body 300 are shown in contact with each other. Each wire comprises an internal conductor 401. Each conductor 401 is surrounded by insulative coating 402. The coating 402 is preferably relatively thin and is robust against mechanical breaches during customary movement and forces after implantation within a patient. Suitable polymers include perfluoroalkoxy (PFA) or polytetrafluoroethylene (PTFE) materials as examples. Other non-polymer based materials could be alternatively employed. By utilizing a suitable non-conductive coating about conductors 401, each conductor 401 is capable of being used as an independent stimulation channel for delivery of electrical pulses to the patient.

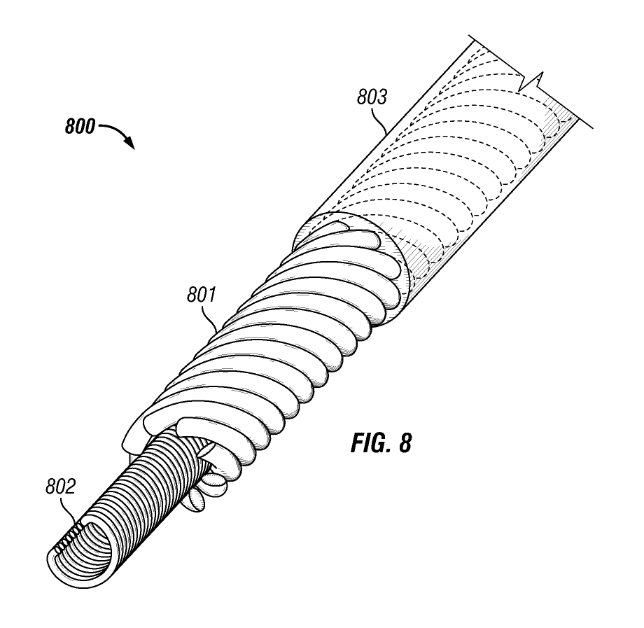

The continuous inner surface produced by tightly wound conductors 401 provides inner lumen 403. Inner lumen 402 may be employed to receive a stylet or guidewire depending upon the intended medical application of lead body 300. A separate structural coil may be inserted within the helical hollow tube (see FIG. 8) whereby the inner lumen will be defined by the inner surface of the separate structural coil. The size of inner lumen 403 may be selected according to the mechanical properties desired for the stylet or guidewire. The size of inner lumen 403 may correspond to the typical inner diameter of traditional “percutaneous” stimulation leads which are currently available, although the inner diameter may be decreased according to other embodiments. The inner diameter of lead body 300 may range from 0.007″ to 0.045″ according to some representative embodiments. In some embodiments, even thought the inner diameter is the same as conventional percutaneous stimulation leads, the outer diameter of lead body 300 may be significantly reduced due to the tight arrangement of the wire strands. In some representative embodiments, the outer diameter may range from 0.011″ to 0.085″.

In the embodiment shown in FIG. 4, eight conductors 401 are shown to support eight independent electrodes (which is a common configuration for neurostimulation leads). However, any suitable numbers of wires and electrodes may be employed for other embodiments such as 4, 8, 16, 32, or more independent wires and electrodes.

FIG. 5 depicts wires of lead body 300 according to one representative embodiment. In the embodiment of FIG. 5, a highly conductive core 502 is provided. For example, silver or silver alloys may be employed for core 502. Such highly conductive materials provide a low resistance path for delivery of current to the patient. However, many common highly conductive materials lack selected mechanical characteristics. Outer layer 501 surrounds core 502. Outer layer 501 may be a different generally conductive metal material with less conductivity than core 502. Outer layer 501 may provide suitable mechanical characteristics to permit integrity of lead body 300 while relatively high contact forces are experienced between neighboring wire strands of lead body 300. Stainless steel materials and Ni-Ti alloy materials may be employed for outer layer 501 as examples. As shown in FIG. 5, outer layer 501 is, in turn, surrounded by insulating layer 402.

FIG. 6 depicts a distal portion of lead body 300. In this case, multiple electrodes 601 are provided directly about the outer diameter of lead body 300. During fabrication, the insulation about a respective conductor 401 at a suitable location at the distal end of lead body 300 is exposed. Laser processing or mechanical processing may be employed as examples. A respective electrode 601 is applied over the exposed conductive material of the conductor 401. The electrode 601 is indirectly or directly conductively coupled to the exposed conductor 401. Terminals (not shown in FIG. 6) may be formed at the proximal end of lead body 300 utilizing substantially the same processing methods. Swaging may be employed to engage lead body 300 with conventional ring electrode components.

In certain embodiments, electrodes 601 are implemented using conductive polymers. For example, intrinsically conductive polymer materials may be employed such as PEDOT:PSS or Poly(3,4-ethylenedioxythiophene) poly(styrenesulfonate) and Ethylenedioxythiophene (EDOT). Compounds of non-conductive polymers with suitable conductive material (e.g., Carbosil™ with silver, platinum, gold, or other conductive materials) could be employed. The conductive polymer may be applied to the outer surface of lead body 300 through micro-inkjet processing or any other suitable process.

In an alternative embodiment, one or more additional insulative coatings may be applied over the entire length of lead body 300 or selective portions of lead body 300. For example, a suitable coating may be applied to the proximal and/or distal end of lead body 300. The suitable coating may provide suitable mechanical characteristics (e.g., to facilitate insertion within a header of an IPG). Also, the coating may provide a beneficial substrate to assist placement of electrodes 601 and/or terminals on lead body 300. The coating may employ any suitable biocompatible material, e.g., PFA, PTFE, polyurethanes, polyether ether ketone (PEEK), etc. Similarly, one or more coatings of such materials may be applied on the inner lumen surface of lead body 300, e.g., to reduce friction by traversal of a guide wire through the inner lumen.

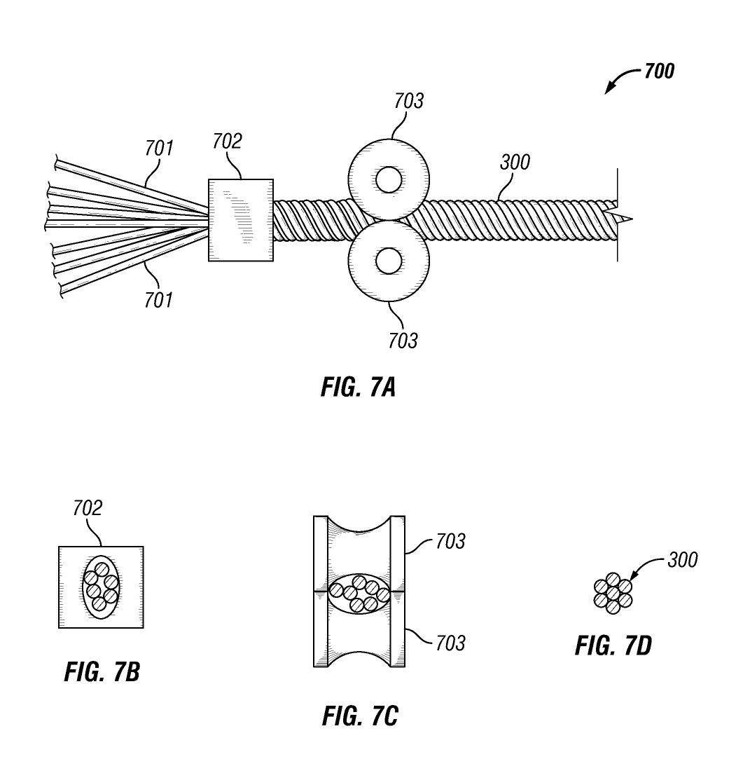

FIG. 7 depicts system 700 for forming lead body by twisting wires along a circular line according to one representative embodiment. Neighboring wires engage tightly with one another without generating a gap therebetween because the neighboring wires are subjected to a compression force when twisted by system 700. As shown in system 700, a plurality of wire strands 701 are initially provided. The wire strands 701 are drawn into vice tool 702 to form a rope structure. In the intermediate rope structure, wire strands 701 are generally oriented in a linear manner and circumscribing an inner axis. One or more inner wires or mandrels may be employed to assist the initial placement of wire strands 701 and the helical tube forming process. Other materials may be employed for the inner wires or mandrels such as softer metals to assist subsequent removal operations. The intermediate rope structure is then rolled between rollers 703 to form the wire-stranded hollow tube structure of lead body 300. The inner wires or mandrels may be removed at any suitable subsequent time. Although system 700 depicts one process for forming lead body 300, any other helical wire forming process now known or later developed may be employed according to other embodiments.

FIG. 8 depicts lead body 800 according to one representative embodiment. Lead body 800 may be utilized to fabricate a stimulation lead using any of the processing discussed herein. Lead body 800 comprises helical hollow tube 801 fabricated as discussed herein. The wire strands of helical hollow tube 801 are electrically isolated from each other as previously discussed. Structural coil 802 is provided within the interior of helical hollow tube 801. Structural coil 802 exhibits substantially greater crush resistance relative to crush resistance of helical hollow tube 801. Structural coil 802 may be provided to function a guide for a stylet. Further, structural coil 802 may be adapted to provide greater tensile strength and provide the required hoop strength to permit swaging of electrodes about lead body 800. During the fabrication of helical hollow tube 801, the wire strands of tube 801 may be directly served over structural coil 802.

Polymer layer 803 is provided over helical hollow tube 801. Polymer layer 803 may provide an additional layer of protection against an electrical short. Further, polymer layer 803 may be adapted to accommodate swaging of electrodes. Any suitable technique may be employed to provide polymer layer 803 such as extrusion and molding processes. Any suitable biocompatible material may be employed for polymer layer including polyurethanes and thermoplastic silicone polycarbonate-urethanes (e.g., CARBOSIL™) as examples.

Although certain representative embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate when reading the present application, other processes, machines, manufacture, compositions of matter, means, methods, or steps, presently existing or later to be developed that perform substantially the same function or achieve substantially the same result as the described embodiments may be utilized. Accordingly, the appended claims are intended to include within their scope such processes, machines, manufacture, compositions of matter, means, methods, or steps.

1. An implantable stimulation lead for delivering electrical stimulation to tissue of a patient, the implantable stimulation lead comprising:
a lead body comprising a plurality of wires strands that form a solid helical hollow tube that is maintained by mutual contact pressure of neighboring wire strands, wherein the plurality of wire strands comprise an outer coating of non-conductive material to electrically isolate each of the plurality of wire strands from each other; and
a plurality of electrodes with each electrode being electrically coupled to one of the plurality of wire strands.

2. The implantable stimulation lead of claim 1 wherein each of the plurality of wire strands comprises a core of highly conductive material and an outer layer of conductive material with high mechanical strength relative of the conductive material of the core.

3. The implantable stimulation lead of claim 2 wherein the highly conductive core substantially comprises silver or a silver alloy.

4. The implantable stimulation lead of claim 2 wherein the conductive material of the outer layer substantially comprises a Ti-Ni alloy.

5. The implantable stimulation lead of claim 1 wherein the outer coating of non-conductive material substantially comprises perfluoroalkoxy (PFA) material.

6. The implantable stimulation lead of claim 1 further comprising:
a structural coil that is disposed within an inner lumen of helical hollow tube that exhibits substantially greater crush resistance relative to crush resistance of the helical hollow tube.

7. The implantable stimulation lead of claim 1 further comprising:
a coating of insulative material about a distal portion of the helical hollow tube and underneath the plurality of electrodes.

8. A method of fabricating an implantable stimulation lead for electrically stimulating tissue of a patient using multiple electrodes, the method comprising: one or more of the steps discussed herein.
